(19) United States
(12) Patent Application Publication
(10) Pub. No.: US 2011/0106180 A1
(43) Pub. Date: May 5, 2011

(54) Implants With Adjustable Saddles

(75) Inventors: Keith E Miller, Germantown, TN (US); Anthony Dickinson, Bartlet, TN (US); Ian Rubin de la Borbolla, Memphis, TN (US); Alan Rozach, Atoka, TN (US); Gary S. Lindemann, Collierville, TN (US); Ryan R. Davis, Cordova, TN (US)

(73) Assignee: Warsaw Orthopedic, Inc., Warsaw, IN (US)

(21) Appl. No.: 12/609,936

(22) Filed: Oct. 30, 2009

(51) Int. Cl. A61B 17/86

(52) U.S. Cl. 606/308

(57) ABSTRACT

A bone anchor assembly is provided, which may be used in cervical, thoracic, lumbar or sacral areas of the spine or other orthopedic locations. The anchor assembly includes a bone engaging portion, a receiver, a saddle within a channel defined by the receiver, and an engaging member. The receiver extends along a central longitudinal axis and is fixed to the bone engaging portion. A rod or other elongated connecting element is received in a channel of the receiver in contact with the saddle, and the engaging member engages the connecting element against the saddle. The orientation of the saddle in the receiver is adjustable to correspond to the orientation of the connecting element relative to the central longitudinal axis at any one of a plurality of angles of the connecting element through the receiver while the receiver and bone engaging portion remain fixed relative to one another.

### BACKGROUND

The present invention concerns bone anchors and anchor assemblies, particularly useful for engagement to vertebrae. In a particular embodiment, the invention contemplates a bone anchor assembly with an adjustable saddle to secure an elongate connecting element, such as a spinal rod, along the spinal column.

Several techniques and systems have been developed for correcting and stabilizing the spine and for facilitating fusion at various levels of the spine. In one type of system, an elongated rod is disposed longitudinally along the length of the spine or several vertebrae of the spinal column. The rod may be bent to correspond to the normal or desired curvature of the spine in the particular region being instrumented. For example, the rod can be bent or angled to form a normal kyphotic curvature for the thoracic region of the spine, or a lordotic curvature for the lumbar region. In accordance with such a system, the rod is engaged to various vertebrae along the length of the spinal column by way of a number of fixation elements. A variety of fixation elements can be provided which are configured to engage specific portions of the vertebra. For instance, one such fixation element is a hook that is configured to engage the lamina of the vertebra. Another type of fixation element is a spinal screw which can be threaded into various aspects of the vertebral bone, such as the pedicle.

In one typical procedure utilizing a bendable, angled or linear rod, one or more of the rods is situated on one or both of the opposite sides of the spine or spinous processes. A plurality of bone screws are threadingly engaged to several vertebral bodies, such as to the pedicles of these vertebrae. One or more of the bone screws are maneuvered to manipulate the position or orientation of the vertebral body or bodies to which the bone screw is engaged. The rod(s) are connected or affixed to the plurality of bone screws to apply and maintain corrective and stabilizing forces to the spine.

The bone anchors in spinal procedures can have receivers with channels for the elongated rod or other member that, in some bone anchors, open upward, i.e. directly away from the bone to which the anchor is attached. Other bone anchors utilize channels that open along the medial or lateral side of the anchor to receive the rod. It is desirable in some procedures to utilize a bone anchor where the bone engaging portion of the bone anchor and the receiver are fixed relative to one another so that the forces applied to the receiver to manipulate the vertebra to which the bone anchor is engaged are effectively transferred to the vertebra. However, the resulting position of the vertebra and the receiver of the bone anchor may require contouring, bending, and/or angling of the rod through the channel of the bone anchor, which can result in a less than optimal fit between the anchor and the rod, creating undesirable stress concentrations in the rod, bone anchor and/or bony structure. Additional improvements in the bone anchor and rod interface in spinal systems are still needed.

### SUMMARY

A bone anchor assembly is provided, which may be used in cervical, thoracic, lumbar or sacral areas of the spine or other orthopedic locations. The anchor assembly includes a bone engaging portion, a receiver, a saddle within a channel defined by the receiver, and an engaging member. The receiver extends along a central longitudinal axis and is immovably fixed to the bone engaging portion. A rod or other elongated connecting element is received in the channel of the receiver in contact with the saddle, and the engaging member engages the connecting element against the saddle. The orientation of the saddle in the receiver is adjustable to correspond to the orientation of the connecting element relative to the central longitudinal axis at any one of a plurality of angles of the connecting element through the receiver while the receiver and bone engaging portion remain fixed relative to one another.

According to a further aspect, a bone anchor assembly for spinal stabilization is provided. The bone anchor assembly includes a distal bone engaging portion and a receiver extending proximally from the bone engaging portion along a central longitudinal axis. The receiver and bone engaging portion form a unitary structure with the receiver including a pair of arms extending along the central longitudinal axis on opposite sides of a channel of the receiver. The receiver includes a bottom surface extending along the channel between the pair of arms, and the channel opens at a proximal end of the pair of arms and the channel opens at opposite sides of the pair of arms. The bone anchor assembly also includes a saddle positioned in the receiver adjacent to the bottom surface of the receiver with the saddle including a proximal support surface. A connecting element extends along a longitudinal axis and is located in the channel and projects through opposite sides of the receiver. The bone anchor assembly also includes an engaging member engaged to the receiver in contact with the connecting element to secure the connecting element against the proximal support surface of the saddle. The saddle moves in a plane defined by the central longitudinal axis of the receiver and the longitudinal axis of the connecting element in response to variation of the connecting element relative to the central longitudinal axis of the receiver from an orthogonal orientation to non-orthogonal orientations.

According to another aspect, a bone anchor assembly for spinal stabilization includes a bone anchor with a proximal receiver and a distal bone engaging portion. The bone anchor assembly includes a saddle mounted in the receiver that is movable in a single plane defined by the central longitudinal axis of the receiver and the longitudinal axis of the connecting element so that a proximal support surface of the connecting element parallels the orientation of the connecting element through the receiver while the receiver and the bone engaging portion are fixed relative to one another.

According to another aspect, a bone anchor assembly includes a distal bone engaging portion and a receiver extending proximally from the bone engaging portion along a central longitudinal axis. The receiver and bone engaging portion form a unitary structure and the receiver defines a channel extending therethrough. A saddle with a proximal support surface is mounted to the receiver in the channel. An elongated connecting element extends along a longitudinal axis through the channel and projects from opposite sides of the receiver. The longitudinal axis of the connecting element and the central longitudinal axis define a plane. The bone anchor assembly also includes an engaging member in contact with the connecting element to secure the connecting element against the proximal support surface of the saddle. The saddle rotates only in the plane to align the support surface with an orientation of the connecting element relative to the central longitudinal axis.

These and other aspects are discussed further below.

### BRIEF DESCRIPTION OF THE FIGURES

FIG. 1 is a posterior elevation view of a spinal column segment with a spinal implant system engaged thereto.

FIG. 2 is an exploded side elevation view of one embodiment of a bone anchor and connecting element.

FIG. 3 is a side elevation view of a bone anchor assembly including the bone anchor and connecting element of FIG. 2 and an engaging member in an exploded relation to the bone anchor.

FIG. 4 is an end elevation view of a saddle of the bone anchor of FIG. 2.

FIG. 5 is an exploded, end elevation view of another embodiment saddle.

FIG. 6 is a side elevation view of a proximal portion of another embodiment bone anchor engageable to the connecting element and engaging member of FIG. 3.

FIG. 7 is an elevation view of another embodiment saddle.

FIG. 8 is a perspective view of the saddle of FIG. 7.

FIG. 9 is a perspective view of a proximal portion of another embodiment bone anchor.

FIG. 10 is a frontal looking cross-section view of the bone anchor of FIG. 9 including an engaging member engaging a connecting element to the bone anchor to form a bone anchor assembly.

FIG. 11 is a plan view of a saddle of the bone anchor of FIG. 9.

FIG. 12 is an end elevation view of the saddle of FIG. 11.

FIG. 13 is a cross-section view of the bone anchor assembly of FIG. 10 along a plane orthogonal to the plane in which the cross-section of FIG. 10 is taken.

FIG. 14 is an exploded, frontal looking cross-section view of a proximal portion of another embodiment bone anchor of the bone anchor assembly of FIG. 9 with another embodiment saddle shown in an insertion orientation proximally of the anchor.

FIG. 15 is a top plan view of the bone anchor of FIG. 14 without the saddle in the receiver.

FIG. 16 is a cross-section view of the saddle of FIG. 14.

FIG. 17 is a laterally looking cross-section of the bone anchor of FIG. 14 showing the saddle in the bone anchor and a connecting element and engaging member engaged to the bone anchor.

FIG. 18 is a side elevation view of another embodiment bone anchor of a bone anchor assembly.

FIG. 19 is a section view of the bone anchor along line 19-19 of FIG. 18.

FIG. 20 is a front elevation view of the bone anchor of FIG. 18.

FIG. 21 is a section view of the bone anchor along line 21-21 of FIG. 20.

FIG. 22 is a front elevation of another embodiment saddle engageable with the bone anchor of FIG. 18.

FIG. 23 is a side elevation view of the saddle of FIG. 22.

FIG. 24 is a section view of the saddle along line 24-24 of FIG. 23.

FIG. 25 is a side elevation view of the other embodiment bone anchor assembly including the anchor of FIG. 18 and saddle of FIG. 22 along with a connecting element and engaging member engaged to the receiver of the bone anchor.

FIG. 26 is a front elevation view of the bone anchor assembly of FIG. 25.

FIG. 27 is a section view of the bone anchor assembly along line 27-27 of FIG. 25.

FIG. 28 is a section view of the bone anchor assembly along line 28-28 of FIG. 26.

FIG. 29 is a frontal sectional view of another embodiment bone anchor with another embodiment saddle.

FIG. 30 is a side sectional view of the bone anchor and saddle of FIG. 29.

FIG. 31 is a frontal section view of another embodiment bone anchor with another embodiment saddle.

FIG. 32 is a bottom plan view of another embodiment of the saddle of FIG. 31.

FIG. 33 is a front elevation view of another embodiment bone anchor.

FIG. 34 is a section view of the bone anchor along line 34-34 of FIG. 33 and further showing the saddle of FIG. 36 engaged to the bone anchor.

FIG. 35 is an enlarged view of a portion B of the section view of the bone anchor of FIG. 34.

FIG. 36 is a plan view of another embodiment saddle engageable with the bone anchor of FIGS. 33-34.

FIG. 37 is a side elevation view of the saddle of FIG. 36.

FIG. 38 is a front elevation view of the saddle of FIG. 36.

FIG. 39 is a front elevation of another embodiment bone anchor and another embodiment saddle in exploded relation to the same.

FIG. 40 is a section view of the bone anchor along line 40-40 of FIG. 39 and further showing the saddle of FIG. 36 engaged to the bone anchor.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein, being contemplated as would normally occur to one skilled in the art to which the invention relates.

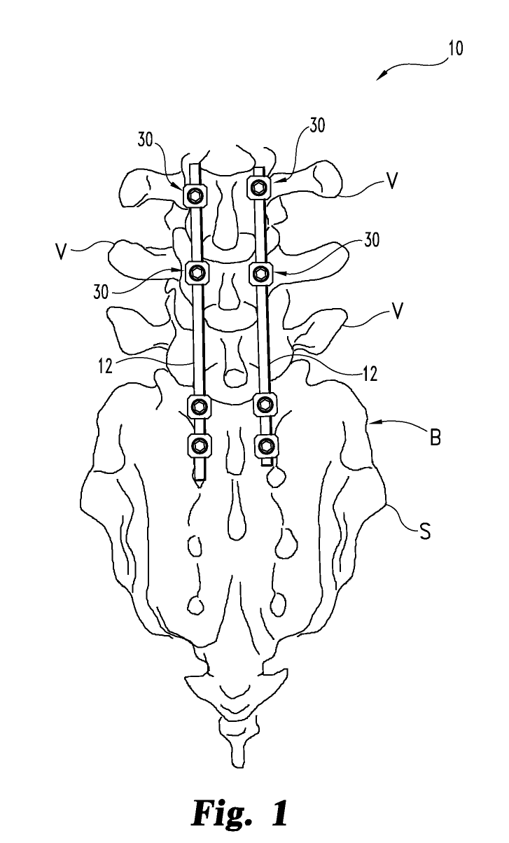

FIG. 1 illustrates a posterior spinal implant system 10 located along a spinal column of a patient. Implant system 10 generally includes several bone anchor assemblies 30 with at least one elongated connecting element 12 structured to selectively interconnect two or more bone anchors. Connecting elements 12 may be a spinal rod, plate, bar, or other elongated element having a length to extend between at least two vertebrae. Spinal implant system 10 may be used for, but is not limited to, treatment of degenerative spondylolisthesis, fracture, dislocation, scoliosis, kyphosis, spinal tumor, and/or a failed previous fusion. More specifically, in one embodiment implant system 10 is affixed to posterior elements, such as the pedicles of vertebra V, or other bones B of the spinal column segment, from a posterior approach. Bones B can include the sacrum S and/or one or more of several vertebrae V. Spinal implant system 10 can be engaged to vertebrae of one or more levels of the sacral, lumbar, thoracic and/or cervical regions of the spinal column. Other embodiments contemplate that spinal implant system 10 is engaged along other portions of the spine, such as the anterior, lateral or oblique portions of the vertebrae V. Still other embodiments contemplate applications in procedures other the spinal stabilization procedures.

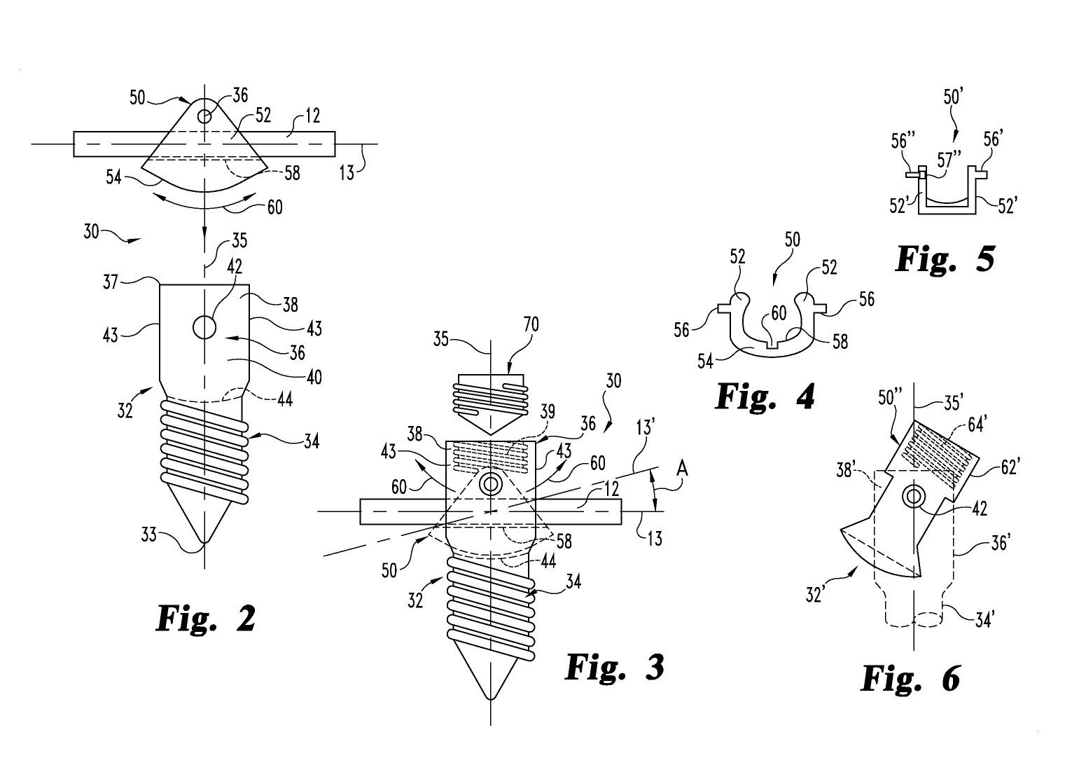

Referring to FIG. 2, there is shown exploded view of one embodiment of bone anchor assembly 30. Bone anchor assembly 30 includes a bone anchor 32 with a distal bone engaging portion 34 configured for attachment to a vertebra, such as cervical, thoracic, lumbar and/or sacral vertebrae, or other bones or tissues in the body of a patient, and a proximal receiver 36. Bone anchor 32 described herein can be included with bone engaging portion 34 configured as a bone screw, vertebral hook, bone clamp, and or other suitable bone engaging arrangement. Bone anchor 32, in the embodiment shown in FIG. 2, includes an elongated bone engaging portion 34 extending from a distal end 33 along a central longitudinal axis 35 to a proximal receiver 36 that also extends along central longitudinal axis 35 to proximal end 37. Bone engaging portion 34 is shown with an elongated shaft having one or more threads along at least a portion thereof. The threads may be cancellous threads with the shaft sized and configured for implantation into a vertebra or other bone. The threads of bone engaging portion 34 may be self-tapping, self-drilling, continuous, intermittent, of multiple thread forms, or other appropriate configurations. Furthermore, bone anchor 32 may include a lumen extending through the proximal and distal ends thereof for receipt of guidewire and/or injection of material into the bone. One or more fenestrations may be provided along bone anchor 32. Bone anchor 32 may also be solid along its length as shown. Receiver 36 extends proximally from and is formed as a unitary, monolithic construct that is fixed with respect to bone engaging portion 34 even before securing connecting element 12 to bone anchor 32. Thus, forces applied to receiver 36 are directly transferred to bone engaging portion 34 and to the bony structure to which bone engaging portion is engaged.

Receiver 36 includes a pair of arms 38 (only one shown) extending along central longitudinal axis 35 on opposite sides of a channel 40 that extends through receiver 36. Channel 40 extends in a generally transverse orientation to arms 38 and bone engaging portion 34 so that connecting element 12 projects outwardly from opposite end openings 43 of channel 40 located at opposite sides of arms 38 of receiver 36 when connecting element 12 is positioned in channel 40, as shown in FIG. 3. Channel 40 includes a bottom surface 44 extending between end openings 43 along the distal ends of arms 38. Each of the arms 38 includes a receptacle 42 extending therein from an inner surface along channel 40 toward an outer surface of the arm 38 in a transverse relationship to central longitudinal axis 35. Each receptacle 42 is for engaging a saddle 50 to receiver 36 with saddle 50 located in channel 40, as also shown in FIG. 3. Each of the arms 38 further includes an internal thread profile 39 extending therealong from a proximal end opening defined by arms 38 at proximal end 37 opposite bone engaging portion 34. The proximal end opening opens into channel 40 in a direction along central longitudinal axis 35, and an engaging member 70 is movably engaged to arms 38 of receiver 36 through the proximal end opening. Engaging member 70 is movable into channel 40 by threading it along arms 38 of receiver 36 to contact connecting element 12 and direct connecting element 12 into receiver 36 and into engagement with saddle 50, which in turn moves and/or forces saddle 50 into contact with bottom surface 44 of anchor 32, securing connecting element 12 and anchor 32 to one another. In the illustrated embodiment, engaging member 70 is a set screw type element with an externally threaded body that threadingly engages inner threads provided along arms 38. Other embodiments contemplate an engaging member in the form of a nut, cap, or combination of nut and set screw. In still other embodiments, engaging member 70 engages receiver 36 in a non-threaded manner, such as a friction fit, interference fit, or bayonet lock.

As shown in FIG. 4, saddle 50 includes a generally U-shaped body with opposite legs 52 and a support member 54 extending between legs 52. When viewed from the side as in FIGS. 2 and 3, legs 52 extend proximally from support member 54 to a proximal end of legs 52. A connector 56 extends outwardly from each of the legs 52 transversely to the width of legs 52 and with connectors 56 extending in opposite directions from one another. Connectors 56 are positioned in receptacle 42 of a respective one of arms 38 to pivotably mount saddle 50 to receiver 36. Support member 54 also includes a support surface 58 between legs 52 against which connecting element 12 is positioned. Legs 52 and support surface 58 form a cradle that receives connecting element 12. Connectors 56 define a pivot axis in receptacles 42. In one embodiment, the pivot axis is located proximally of the longitudinal axis 13 of connecting element 12 while supporting support member 54 in a spaced relationship to bottom surface 54 of receiver 36, allowing pivotal movement of the connecting element 12 and saddle 50 in receiver 36 as connecting element 12 is moved into non-orthogonal orientations to central longitudinal axis 35. In a further embodiment, the pivot axis defined by the connection of connectors 56 with receiver 36 is located proximally of the proximal side of connecting element 12. The location of the pivot axis provides a wide range of pivoting motion of saddle 50 relative to receiver 36. When attached to receiver 36, saddle 50 pivots relative to arms 38 about connectors 56, as indicated by arrows 60, as dictated by the angle of connecting element 12 relative to central longitudinal axis 35. In FIG. 3, connecting element 12 and its longitudinal axis 13 are shown in an orthogonal orientation to central longitudinal axis 35. As indicated by longitudinal axis 13′ and arrows 60, saddle 50 and connecting element 12 are pivotal relative to receiver 36 about the pivot axis defined by connectors 56 and their engagement with receiver 36 at an angle A with the orthogonal orientation. In one embodiment, angle A ranges from 0 degrees to about ±90 degrees. In another embodiment, angle A ranges from 0 degrees to about ±15 degrees.

Saddle 50 is movably positioned in channel 40 of receiver 36 so that saddle 50 pivots or rotates in a plane defined by central longitudinal axis 35 of receiver 36 and longitudinal axis 13 of connecting element 12. In one embodiment, to facilitate its assembly with receiver 36, at least a portion of saddle 50 is flexible and resilient so that the proximal ends of legs 52 can be moved toward one another, thereby displacing connectors 56 inwardly as legs 52 are displaced inwardly, allowing connectors 56 to be positioned in channel 40 in alignment with a respective one of the receptacles 42. The inward force on legs 52 is released to allow legs 52 to spring or deflect toward their undeflected position, positioning connectors 56 into receptacles 42. A notch or other relief structure 60 can be formed in support surface 58 to facilitate bending of support member 54 and thus the inward deflection of legs 52 and connectors 56, such as shown in FIG. 4, for positioning in saddle 50 in channel 40 of receiver 36 between arms 38. In other embodiments, relief structure 60 is located in the surface of support member 54 opposite support surface 58. Another embodiment saddle 50′ is shown in FIG. 5. In this embodiment, saddle 50′ includes a stationary connector 56′ on one side and a spring-assisted connector 56″ on the opposite side. The spring-assisted connector 56″ is displaced inwardly into receptacle 57″ of leg 52′ to allow insertion of connector 56″ and connector 56′ into receiver 36 between arms 38. Spring assisted connector 56″ is spring-biased laterally outwardly and into the adjacent receptacle 42 of receiver 36 when it is positioned in axial alignment therewith.

Referring now to FIG. 6, another embodiment anchor 32′ includes a bone engaging portion 34′ and receiver 36′. Receiver 36′ differs from receiver 36 discussed above in that arms 38′ are not configured to engage engaging member 70. Rather, saddle 50″ includes a proximal extension 62′ from each leg 52′. Extensions 62′ include thread profile 64′ on the internal and/or external surfaces thereof to engage engaging member 70. In this embodiment of the saddle and receiver, engaging member 70 remains in a substantially orthogonal orientation to longitudinal axis 13 of connecting element 12 as is it moved in engagement with saddle 50″. In configurations where the orientation of longitudinal axis 13 of connecting element 12 is non-orthogonal to central longitudinal axis 35′ of bone anchor 32′, engaging member 70 moves along saddle 50″ in an orthogonal orientation to longitudinal axis 13 and in an oblique orientation to central longitudinal axis 35′. Saddle 50″ and connecting element 12 pivot in receiver 36′ in a plane defined by longitudinal axis 13 and central longitudinal axis 35′. In the embodiment of receiver 36 and saddle 50 discussed above, engaging member 70 is advanced along central longitudinal axis 35 toward connecting element 12 regardless of the orientation of longitudinal axis 13 of connecting element 12 relative to longitudinal axis 35 since saddle 50 and connecting element 12 pivot in receiver 36 in a plane defined by longitudinal axis 13 and central longitudinal axis 35.

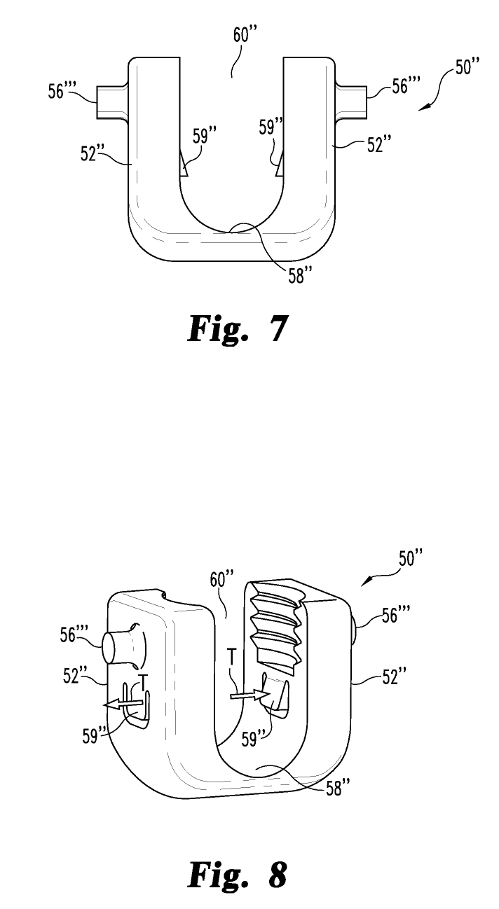

FIGS. 7-8 show further details of saddle 50″. Saddle 50″ includes legs 52″ that define inner support surface 58″ and connectors 56′″ extending outwardly from legs 52″ to mount saddle 50′″ with receptacles 42 of receiver 36′. Legs 52″ also each include a tab 59″ projecting inwardly into the channel 60″ between legs 52″ that receives connecting element 12. As connecting element 12 is pushed into channel 60″ toward support surface 58″ by the engaging member 70 being advanced along the threads of legs 52″, tabs 59″ are pushed outwardly as indicated by arrows T. Tabs 59″ are configured to project outwardly from the outer sides of legs 52″ and press against receiver 36′ to lock saddle 50″ in its angled position relative to receiver 36′.

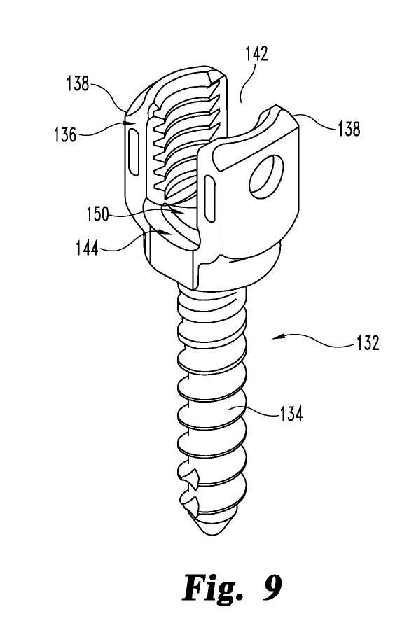

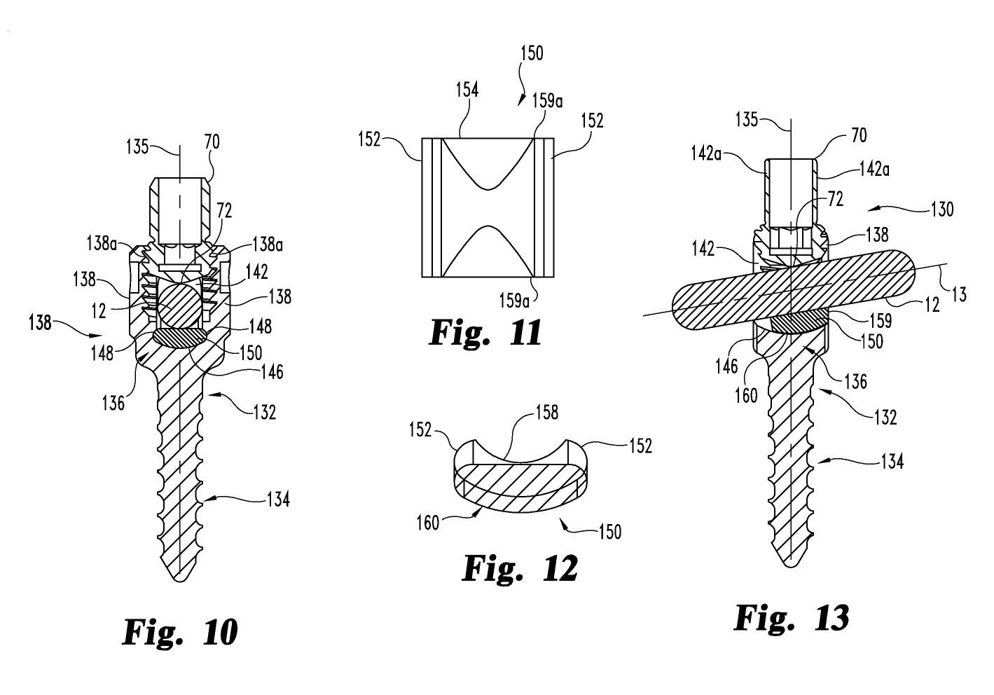

Referring to FIGS. 9-13, another embodiment anchor assembly 130 is shown that includes an anchor 132 and a saddle 150. Anchor 132 includes a bone engaging portion 134, which can include any of the features of bone engaging portion 34 discussed above. A receiver 136 extends proximally from bone engaging portion 134 along a central longitudinal axis 135, and can include any of the features of receiver 36 discussed above. Engaging member 70 is engageable to receiver 136. Receiver 136 is fixed relative to bone engaging portion 134, and forms a monolithic bone anchor 132 made from a single piece of material or multiple components in which the portions of bone anchor 132 are rigidly connected to one another. In this embodiment of bone anchor 132, receiver 136 includes a bottom surface 144 along channel 142 with an undercut 146 structurally configured to receive and constrain saddle 150 in receiver 136 along channel 142. Arms 138 include inner surfaces 138a that overhang undercut 146 to form lips 148 in channel 142 to capture saddle 150 in receiver 136.

As shown in FIGS. 11 and 12, saddle 150 includes a support member 154 with a proximally oriented support surface 158. Legs 152 extend proximally from support surface 158 on each side of saddle 150 to engage the adjacent lip 148 along each of the arms 138 and retain saddle 150 in undercut 146 while allowing saddle 150 to pivot in receiver 136 to follow the angle connecting element 12 through receiver 136. Saddle 150 includes a distal surface 160 that is convexly curved between arms 152 and convexly curved between opposite between ends 159, providing a bowl shaped distally oriented surface to facilitate pivoting movement of saddle 150 in undercut 146. As shown in FIG. 13, connecting element 12 extends along a longitudinal axis 13, and saddle 150 pivots in receiver 136 so that connecting 12 and its longitudinal axis 13 can be oriented in orthogonal and oblique orientations relative to central longitudinal axis 135 in a plane defined by axes 13, 135. In one embodiment, the angulation of connecting element 12 and its longitudinal axis 13, and thus the orientation of support surface 158 between ends 159 of saddle 150, can vary up to 45 degrees from an orthogonal orientation to central longitudinal axis 135. In one embodiment, engaging member 70 includes a tapered tip 72 to allow connecting element 12 to be pivoted in receiver 136 about tip 72 even when tip 72 is initially in contact with connecting element 12. Engaging member 70 can be further moved to firmly engage or penetrate connecting element 12 with tip 72 to fix connecting element 12 and saddle 150 in position in receiver 136.

Saddle 150 is assembled with receiver 136 by orienting legs 152 toward the opposite end openings 142a of channel 142. Saddle 150 is then positioned in channel 142 in this orientation until it is adjacent to undercut 146, and then saddle 150 is rotated 90 degrees so that legs 152 are aligned under the adjacent lip 148 to pivotably capture saddle 150 in receiver 136. Saddle 150 can then be retained in receiver 136 merely by lips 148, or by pivotally fixing saddle 150 in receiver 136 with a stake, swage, laser weld, or flexible retaining member, for example, to prevent saddle 150 from rotating around central longitudinal axis 135 back to its insertion orientation where legs 152 are aligned with the side openings 142a of channel 142, while permitting ends 159 of saddle 150 to pivot distally and proximally relative to central longitudinal axis 135 to accommodate oblique orientations of connecting element 12 relative to central longitudinal axis 135 through end openings 142a of receiver 136.

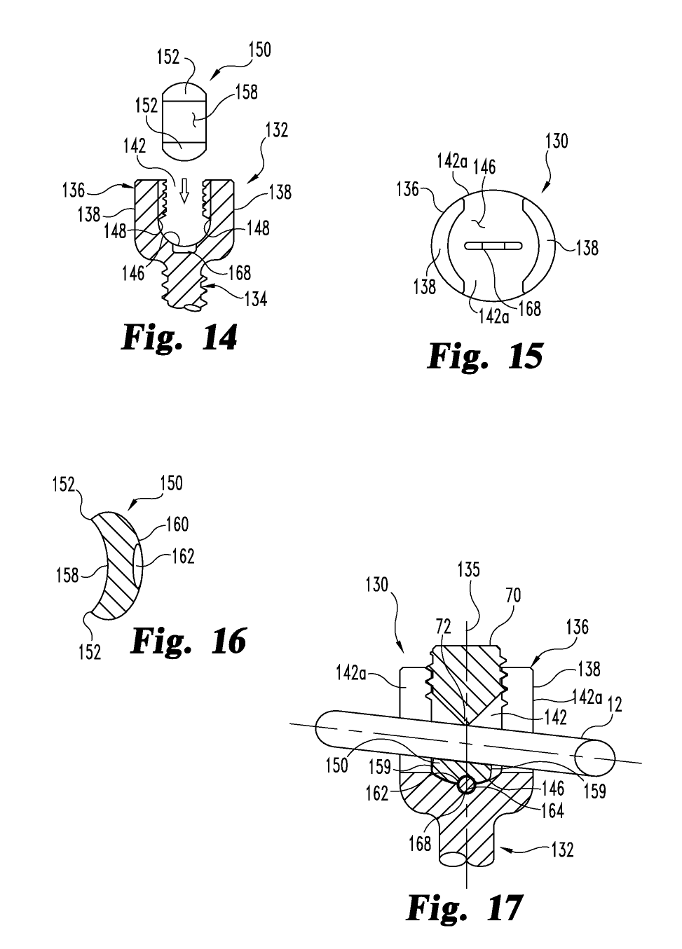

FIGS. 14-17 show an embodiment of the bone anchor assembly 130 with a flexible retaining member 164 that pivotally retains saddle 150 in receiver 136. Receiver 136 includes a slot 168 in undercut 146 in communication with channel 142. Slot 168 extends in the direction toward end openings 142a of channel 142, and houses retaining member 164 therein. In one embodiment, retaining member 164 is a wire made from nitinol or other suitable spring material with opposite ends engaged at the respective opposite ends of slot 168. Distal surface 160 of saddle 150 includes a groove 162 that accepts the convexly curved middle portion of retaining member 164 between the ends in slot 168. The engagement between retaining member 164 and saddle 150 substantially prevents rotation of saddle 150 about central longitudinal axis 135 while allowing ends 159 of saddle 150 to pivot distally and proximally relative to central longitudinal axis 135 in a plane defined by axes 135 and 13 to accommodate oblique orientations of connecting element 12 through end openings 142a of receiver 136, as shown in FIG. 17.

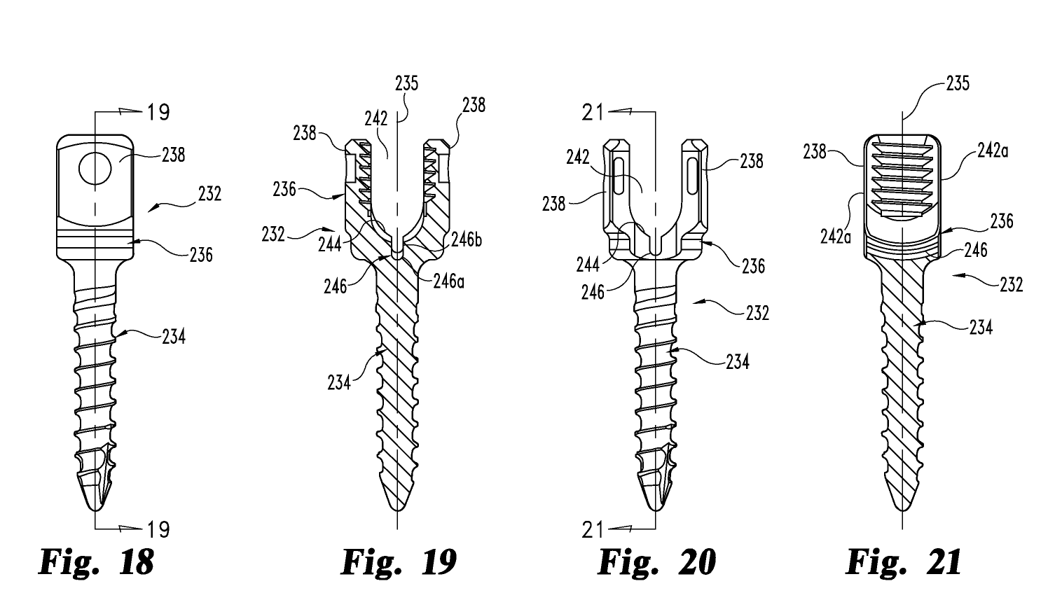

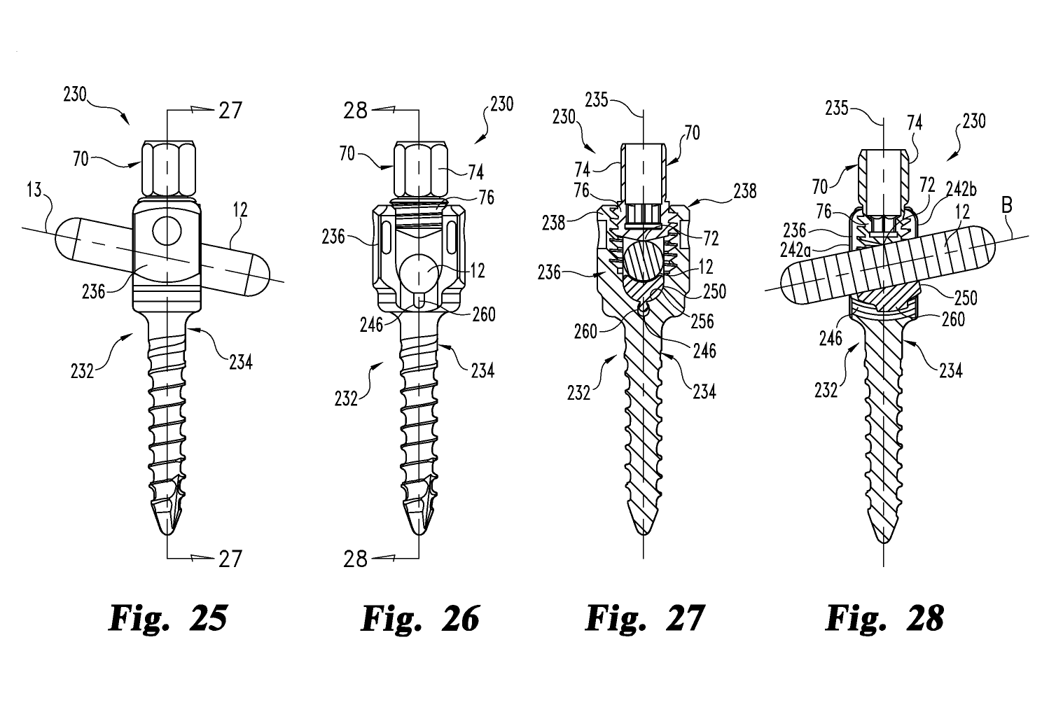

Referring now to FIGS. 18-21, another embodiment anchor 232 is shown for use with anchor assembly 230 of FIGS. 25-28. Anchor 232 includes a distal bone engaging portion 234 and a proximal receiver 236 extending along a central longitudinal axis 235. Except as otherwise discussed herein, anchor 232 can include any one or all of the features and variations of the other anchor embodiments discussed herein. Receiver 236 includes opposite arms 238 extending proximally and distally along central longitudinal axis 235. A channel 242 extends between arms 138, and opens at the proximal ends of arms 238 to receive engaging member 70. A bottom surface 244 of receiver 236 extends between arms 238 along a distal side of channel 242. A slot 246 is formed in receiver 236 along bottom surface 244. Slot 246 extends between opposite sides 242a of receiver 236. Connecting element 12 exit channel 42 of receiver 236 at opposite sides 242a. As shown in FIG. 19, slot 246 includes a bulbous or enlarged distal portion 246a, and a narrower proximal portion 246b that opens into channel 242. Proximal portion 246b assists in proximally retaining saddle 250 in slot 246, as discussed further below.

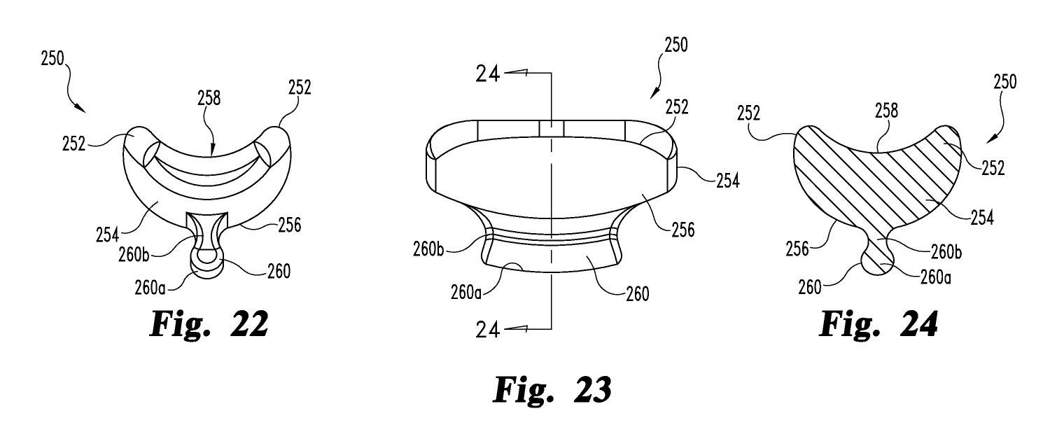

Referring to FIGS. 22-24, another embodiment saddle is shown and designated at 250. Saddle 250 is engageable to and movable in channel 242 of receiver 236 of anchor 232 discussed above. Saddle 250 includes a support member 254 extending between opposite legs 252. The proximal side of saddle 250 includes a support surface 258 that is concavely curved or includes any other suitable configuration to receive connecting element 12 thereagainst. Saddle 250 also includes a bowl shaped convex distal surface 256 opposite support surface 258. Saddle 250 also includes a fin 260 extending distally from distal surface 256. Fin 260 includes an enlarged bulbous distal end portion 260a and a proximal neck portion 260b extending between support member 254 and distal end portion 260a.

As shown in FIGS. 25-28, saddle 250 is positioned in channel 242 of receiver 236 with fin 260 received in slot 246 and distal surface 256 abutting bottom surface 244 of receiver 236. Distal end portion 260a of fin 260 is received in distal portion 246a of slot 246, and neck portion 260b is received in proximal portion 246b of slot 246. Neck portion 246b prevents enlarged distal end portion 260a of fin 260 from passing proximally through slot 246, capturing saddle 250 in slot 246. Distal portion 246a of slot 246 includes a depth greater than the proximal-distal height of distal end portion 260a of fin 260 to provide clearance for fin 260 to facilitate translation along slot 246.

Slot 246 is elongated and extends between opposite sides 242a of arms 238 of receiver 236, allowing saddle 250 to translate between sides 242a by fin 260 moving along slot 246. Furthermore, slot 246 extends along an arc between sides 242a so that the proximal support surface 258 of saddle 250 changes its orientation relative to central longitudinal axis 235 of receiver 236 as it translates along slot 246. One or both of the ends of slot 246 adjacent to sides 242a may include a wall or blind end to prevent saddle 260 from exiting slot 246. In one embodiment, one end of slot 246 includes a blind end or wall, while the opposite end is open to allow insertion of fin 260 into slot 246. The open end of slot 246 is thereafter swaged, deformed, staked, plugged or otherwise obstructed to retain fin 260 in slot 246.

Saddle 250 can be adjusted by sliding or translating it in receiver 236 in or along a plane that includes central longitudinal axis 235 of receiver 236 and longitudinal axis 13 of connecting element 12 to adjust support surface 258 to accommodate non-orthogonal orientations of connecting element 12 relative to central longitudinal axis 235 while maintaining all or substantially all of support surface 258 in contact with connecting element 12. Anchor 232 is employed in anchor assembly 230 with connecting element 12, saddle 250 and engaging member 70 to allow uni-planar angular adjustment of connecting element 12 through receiver 236 of anchor 232. Engaging member 70 is engaged between arms 238 with its distal end 72 in contact with connecting element 12 to secure it against saddle 250 and to fix connecting element 12 in positioned relative to bone anchor 232. In the illustrated embodiment, engaging member 70 includes a proximal break-off portion 74 which is severed from body 76 of engaging member 70 upon application of a torque exceeding a threshold torque. Engaging members without a break-off portion, such as shown above, are also contemplated.

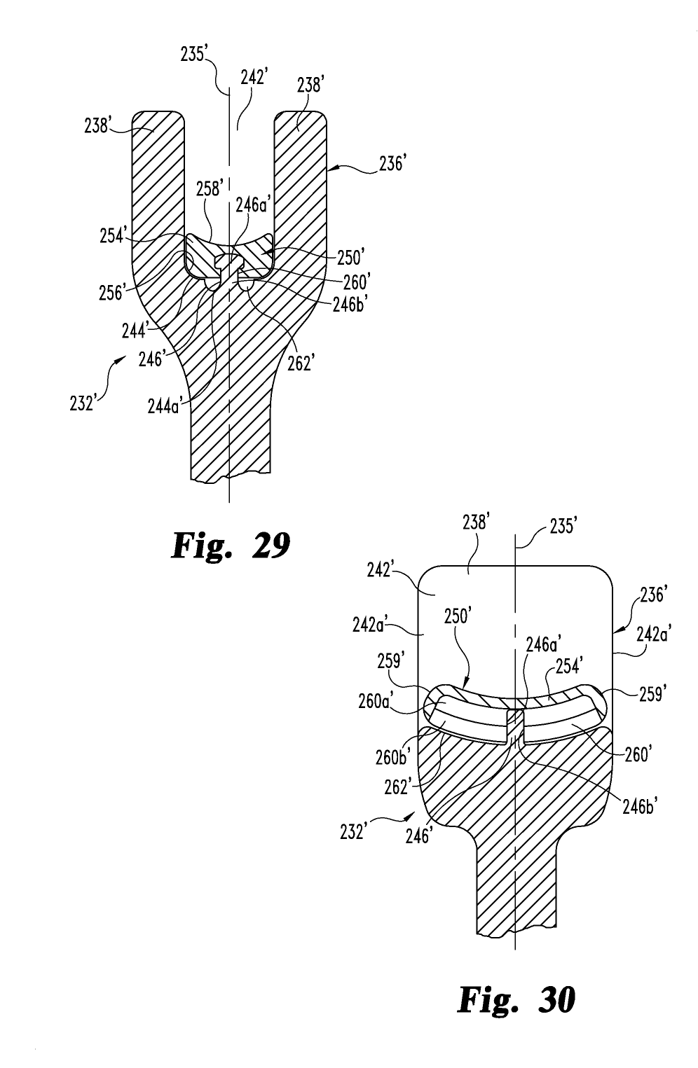

Referring now to FIGS. 29-30, another embodiment of anchor 232′ is shown that is the same as anchor 232 except as otherwise discussed below. Anchor 232′ is employed in an anchor assembly with connecting element 12, saddle 250′ and engaging member 70 to allow uni-planar angular adjustment of connecting element 12 through a receiver 236′ of anchor 232′. Anchor 232′ includes receiver 236′ defining a channel 242′ between arms 238′. A bottom surface 244′ extends between arms 238′. A post 246′ extends proximally from bottom surface 244′ into channel 242′ along central longitudinal axis 235′ of receiver 236′. There is further provided a saddle 250′ that is mounted to post 246′. Saddle 250′ includes a support member 254′ with a proximal support surface 258′ and a distal surface 256′. A slot 260′ is formed through distal surface 256′ into support member 254′. Slot 260′ includes an enlarged proximal portion 260a′ to receive an enlarged proximal head 246a′ of post 246′, and a narrower distal neck portion 260b′ to receive a narrow stem portion 246b′ of post 246′. As shown in FIG. 30, slot 260′ is elongated and arced in the direction between ends 259′ of saddle 250′ adjacent to end openings 242a′ of channel 242′. Saddle 250′ can translate, as indicated by arrows 251′, between end openings 242a′ along the arced translation path so that support surface 258′ can pivot or rotate to accommodate non-orthogonal orientations of connecting element 12 through channel 242′. Bottom surface 244′ may also include an undercut area 244a′ around post 246′ to allow for locking of saddle 250′ in channel 242′.

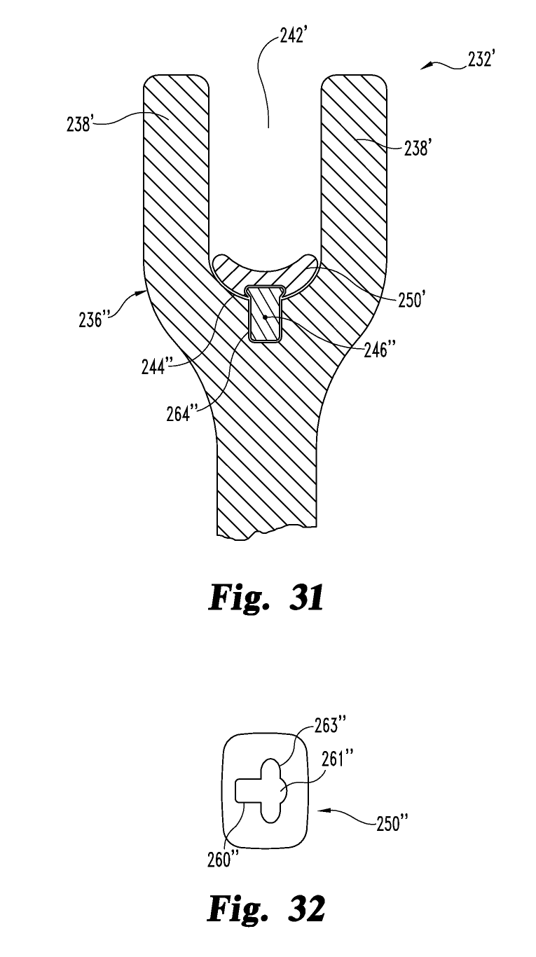

FIG. 31 shown another embodiment of anchor 232′ with a receiver 236″ that includes a post 246″ formed as a separate member that is staked into an opening 264″ in bottom surface 244″ of receiver 236″. Otherwise, saddle 250′ is slidably mounted to post 246″ in a manner similar to that discussed above with respect to post 246′ and saddle 250′. FIG. 32 shows a bottom view of another embodiment saddle 250″ that is similar to saddle 250′, but includes a slot 260″ with a transverse widened end portion 263″ intersecting one end of the primary translation slot 260″. The transverse end portion 263″ allows saddle 250″ to be top loaded through the proximal end opening of channel 242′ and moved through receiver 236′ in an orientation rotated 90 degrees from its final implantation orientation. Once post 246′ is positioned into slot 260″ at widened end portion 263″, saddle 250″ is rotated 90 degrees to align primary translation slot 260″ in the direction between ends 242a′ of receiver 236′ in its implantation orientation.

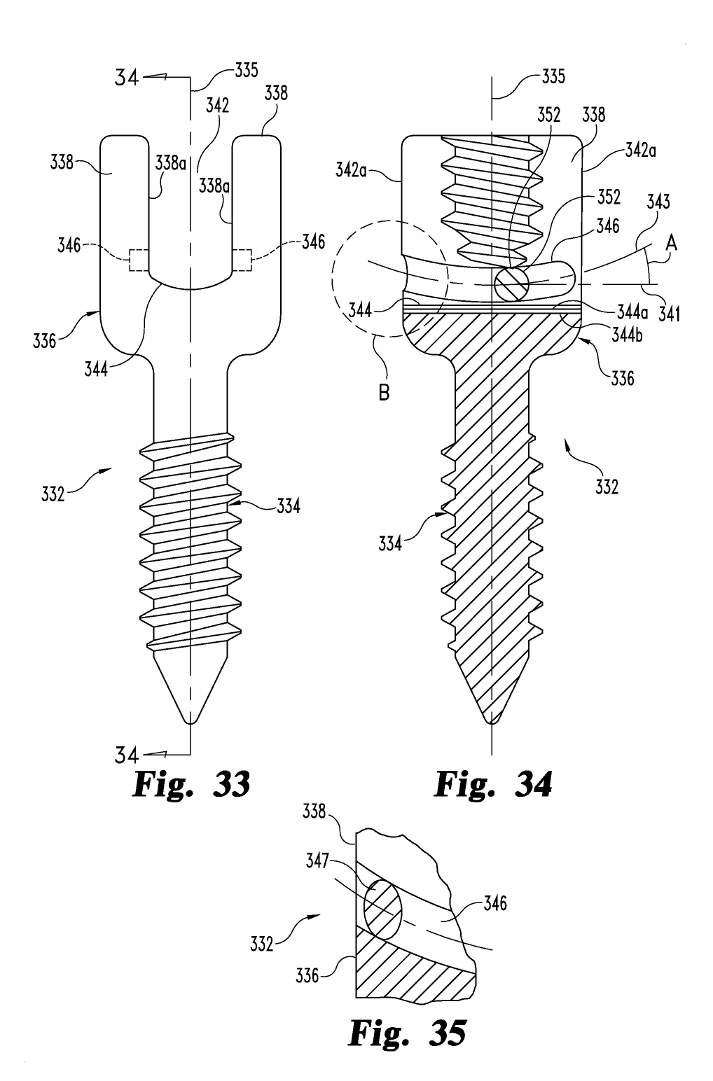

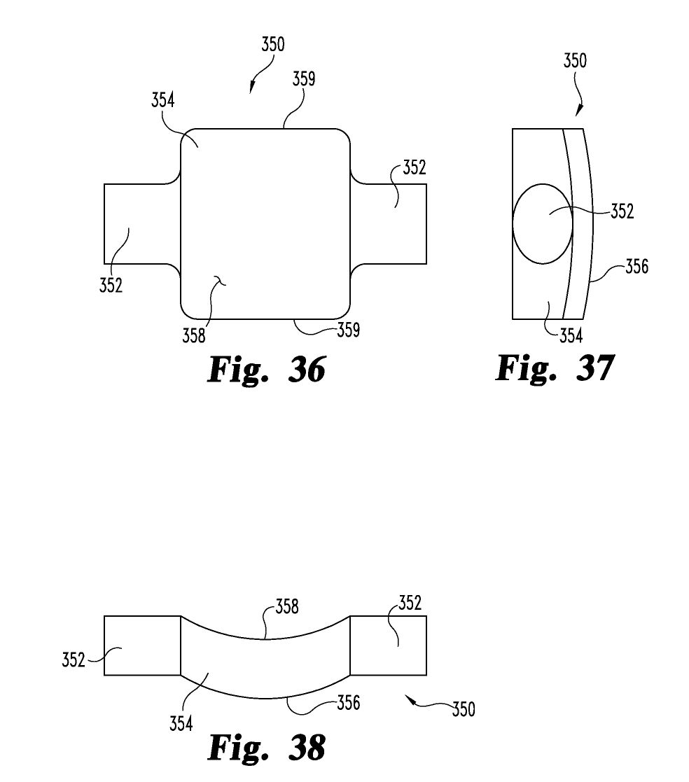

Referring now to FIGS. 33-35, another embodiment anchor 332 is shown that receives another embodiment saddle 350, shown in FIG. 36-38. Anchor 332 includes a bone engaging portion 334 and a receiver 336 extending along a central longitudinal axis 335. An engaging member, such as engaging member 70 discussed herein, is engageable to receiver 336 to secure connecting element 12 in a channel 342 of receiver 336 with the connecting element extending from opposite sides 342a of receiver 336. Except as otherwise discussed, anchor 332 can include any of the features of the anchor embodiments discussed herein, and is employed in an anchor assembly with connecting element 12, saddle 350 and engaging member 70 to allow uni-planar angular adjustment of connecting element 12 through receiver 336.

Receiver 336 includes opposite arms 338 extending along central longitudinal axis 335, and channel 342 is located between arms 338 between opposite end openings 342a. Channel 342 also opens at the proximal end of arms 338, and is configured to receive engaging member 70 through the proximal end opening into channel 342 defined between arms 338. Receiver 336 includes a bottom surface 344 that extends between arms 338. Bottom surface 344 includes a stepped configuration to provide a rigid interface with saddle 350 when saddle 350 is pressed against the stepped region by engaging member 70 pressing against connecting element 12 in channel 342. In the illustrated embodiment, bottom surface 344 includes opposite intermediate stepped regions 344a and a central stepped region 344b that together form a number of elongated ridges extending along bottom surface 344 between arms 338 that grip or bite into the distal surface of saddle 350 when it is pressed against the ridges.

Anchor 332 further includes a translation slot 346 formed in the inner surface 338a of each of the arms 338. Translation slots 346 are elongated in the direction toward end openings 342a of channel 342, and provide a path along which saddle 350 translates in receiver 336. As shown in FIG. 34, slot 346 includes an arced configuration along the width of arm 338 between end openings 342a. There is shown a horizontal datum 341 that is tangential to the distal side of slot 346 at central longitudinal axis 335. Slot 346 diverges from horizontal datum 341 along a translation path 343 at an angle A in the direction away from central longitudinal axis 335 toward opposite end openings 342a. Angle A may be an angle ranging from 0 degrees to 45 degrees with respect to horizontal datum 341. In one embodiment, angle A ranges from 0 degrees to about 15 degrees. One of the ends of slots 346 adjacent to an end opening 342a includes a blind wall, such as shown in FIG. 34, to retain saddle 350 in slot 346. The other end of slots 346 may be open to facilitate placement of saddle 350 into slots 346. As shown in FIG. 35, a locking component 347, such as a pin, wedge, block, swage or other device or deformation of receiver 336 can be placed into the end opening of slot 346 to prevent saddle 350 from exiting therethrough after saddle 350 is assembled with receiver 336.

FIGS. 36-38 show saddle 350 with a central support member 354 and opposite legs 352 extending from opposite sides of support member 354. Support member 354 extends between ends 359 that are oriented toward respective ones of the end openings 342a when saddle 350 is positioned in receiver 336. Support member 354 includes a proximal support surface 358 that is concavely curved or otherwise configured to match the configuration of the outer surface of connecting element 12 to provide a supporting relationship therewith. Support member 354 also includes a distal surface 356 opposite support surface 358 that is convexly curved to engage and translate along bottom surface 344 of receiver 336. In the illustrated embodiment, legs 352 extend laterally from opposite sides of support member 354 and are received in respective ones of the slots 346 along legs 338, as shown in FIG. 34. Legs 352 include a circular or rounded cross-section to facilitate sliding movement and translation along slots 346. The geometry of legs 352 mates in the arced slots 346 to allow for translation along the translation path 343. Legs 352 provide ears or rounded bosses extending from support member 354, although other geometries with slot 346 are also contemplated, including dovetail joints, rectangular interfaces, triangular interfaces, hexagonal interfaces, and multi-angular interfaces between legs 352 and slots 346.

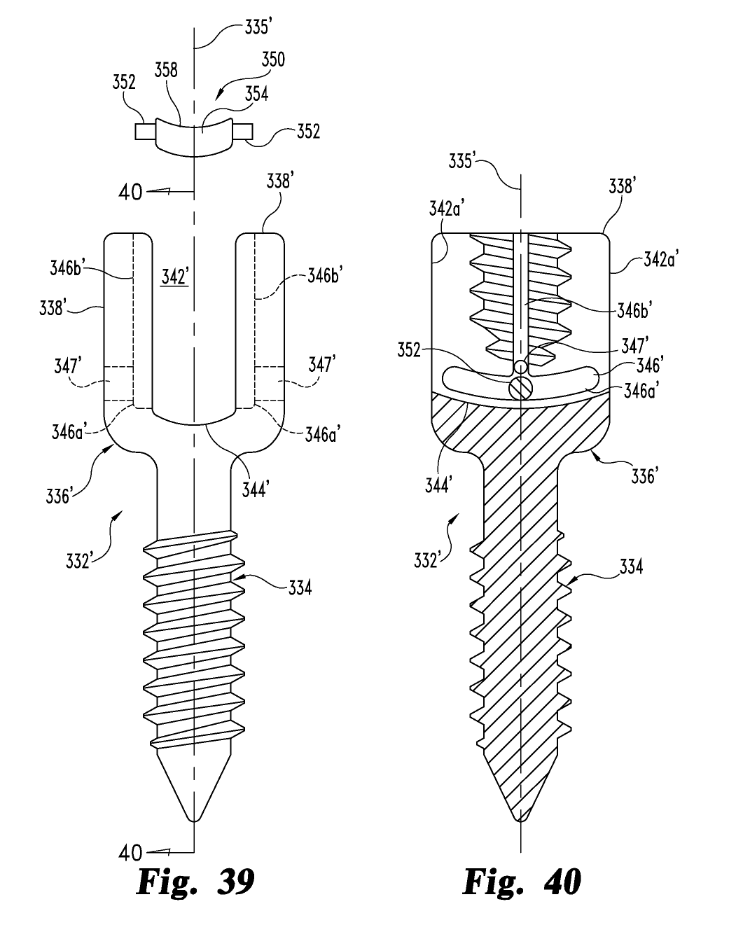

FIGS. 39-40 show another embodiment receiver 336′ with a channel 342′ and bottom surface 344′. Receiver 336′ includes a modified version of slot 346′ where both ends of slot 346′ include a blind end adjacent to end openings 342a′ of channel 342′. In order to position saddle 350 in receiver 336′, arms 338′ include a longitudinal slot portion 346b′ extending proximally from translation slot portion 346a′ through the proximal ends of arms 338′. Longitudinal slot portions 346b′ receive ears 352 of saddle 350 and allow saddle 350 to slide distally along central longitudinal axis 335′ and arms 338′ until ears 352 are located in translation slot portions 346a′, as shown in FIG. 40. Each of arms 338′ includes a bore 347′ extending therethrough that open in longitudinal slot portions 346b′, and bores 347′ receive a locking component such as a pin, screw or other blocking device that obstructs longitudinal slot portions 346b′ and prevents saddle 350 from passing therethrough from translation slot portion 346a′.

Materials for the anchors, saddles and engaging members disclosed herein can be chosen from any suitable biocompatible material, such as titanium, titanium alloys, or other suitable metal or non-metal material. Connecting element 12 can be made from the same material as one or more of the components of the anchor assembly to which it is engaged, or from a different material. For example, connecting element 12 can be made from PEEK, plastic, titanium or titanium alloy, cobalt-chrome, composite material, or other material that is the same or different from the material of one or more components of the anchor assembly to which is engaged. The anchor assemblies can be sized for placement at any level of the spine and for engagement with any bony portion of the spine. In one particular embodiment, the anchor assemblies are engaged to pedicles of the vertebrae. Of course, it is understood that the relative size of the components of the anchor assemblies can be modified for the particular vertebra(e) to be instrumented and for the particular location or structure of the vertebrae to which the anchor assembly will be engaged.

While the invention has been illustrated and described in detail in the drawings and foregoing description, the same is to be considered as illustrative and not restrictive in character. All changes and modifications that come within the spirit of the invention are desired to be protected.

What is claimed is:

1. A bone anchor assembly, comprising:
a distal bone engaging portion;
a receiver extending proximally from said bone engaging portion along a central longitudinal axis, wherein said receiver and said bone engaging portion are a rigid structure, said receiver including a pair of arms extending along said central longitudinal axis on opposite sides of a channel and a bottom surface extending along said channel between said pair of arms, wherein said channel opens at a proximal end of said pair of arms and said channel opens at opposite sides of said pair of arms;
a saddle positioned in said channel of said receiver adjacent to said bottom surface of said receiver, said saddle including a proximal support surface;
a connecting element extending along a longitudinal axis, said connecting element being located in said channel and projecting through said opposite sides of said receiver; and
an engaging member in contact with said connecting element to secure said connecting element against said proximal support surface of said saddle, wherein said saddle moves in a plane defined by said central longitudinal axis of said receiver and said longitudinal axis of said connecting element in response to variation of said connecting element relative to said central longitudinal axis of said receiver from an orthogonal orientation to non-orthogonal orientations while said receiver and said bone engaging portion are fixed relative to one another.

2. The bone anchor assembly of claim 1, wherein said non-orthogonal orientations vary up to 45 degrees from said orthogonal orientation.

3. The bone anchor assembly of claim 1, wherein:
said pair of arms include inner surfaces facing one another on opposite sides of said channel;
said inner surfaces each include an arcuate slot portion formed therein that extends transversely to said central longitudinal axis between said opposite sides of said pair of arms; and
said saddle includes a support member defining said proximal support surface and a pair of legs extending from opposite sides of said support member, said legs being positioned in and slidably movable along a respective one of said arcuate slot portions.

4. The bone anchor assembly of claim 3, wherein said inner surfaces of said pair of arms each includes a longitudinal slot portion therein and said longitudinal slot portions each extend from a proximal end of said respective arm to said arcuate slot portion of said respective arm.

5. The bone anchor assembly of claim 1, wherein:
said receiver includes an elongate, arcuate slot formed in said bottom surface that extends in a direction toward where said channel opens at said opposite sides of said pair of arms; and
said saddle includes a support member defining said proximal support surface, said support member further including a distal surface opposite said proximal support surface and a fin extending distally from said distal surface that is positioned in and slidable along said arcuate slot of said receiver to vary an orientation of said proximal support surface in accordance with a position of said fin along said arcuate slot.

6. The bone anchor assembly of claim 5, wherein:
said arcuate slot includes an enlarged distal portion and a narrower proximal portion extending between said distal portion and said channel;
said fin includes a bulbous distal end in said enlarged distal portion of said arcuate slot and a neck portion extending between said bulbous distal end and said distal surface, said neck portion being positioned in said narrower proximal portion of said arcuate slot; and
said enlarged distal portion of said slot includes a length in a direction along said central longitudinal axis that is substantially greater than a length of said bulbous distal end of said fin along said central longitudinal axis.

7. The bone anchor assembly of claim 1, wherein:
said saddle includes a convexly curved distal surface opposite said proximal support surface, said distal surface including an elongate, arcuate slot formed therein oriented in a direction between said opposite sides of said pair of arms; and
said receiver includes a post extending proximally from said bottom surface that is positioned in said arcuate slot of said saddle and said saddle is slidable along said post to vary an orientation of said proximal support surface in accordance with a position of said saddle along said post.

8. The bone anchor assembly of claim 7, wherein:
said arcuate slot includes an enlarged proximal portion and a narrower distal portion extending between said distal portion and said distal surface of said saddle;
said post includes a bulbous proximal end in said enlarged proximal portion of said arcuate slot and a neck portion extending between said bulbous proximal end and said bottom surface, said neck portion being positioned in said narrower distal portion of said arcuate slot; and
said enlarged proximal portion of said slot includes a length in a direction along said central longitudinal axis that is substantially greater than a length of said bulbous proximal end of said post along said central longitudinal axis.

9. The bone anchor assembly of claim 1, wherein:
said receiver includes an undercut in said bottom surface and said pair of arms each include an inner surface facing said channel, said inner surfaces each defining a lip extending into said channel overhanging said undercut; and
said saddle includes a support member defining said proximal support surface and a pair of legs extending from opposite sides of said support member, said legs being located distally of said lips so that said lips capture said saddle in said undercut.

10. The bone anchor assembly of claim 9, wherein:
said saddle member includes a distal surface opposite said proximal support surface and a groove in said distal surface;
said bottom surface of said receiver includes a receptacle; and
a retaining member located in said receptacle and in said groove, said retaining member normally biasing said saddle member proximally toward said channel.

11. The bone anchor assembly of claim 1, wherein:
said saddle includes a generally U-shaped body with opposite legs and a support member extending between said legs, wherein said legs extend proximally from said support member to a proximal end of said legs;
a connector extending outwardly from each of said legs in opposite directions from one another; and
said receiver includes a receptacle in each of said arms and said connectors are positioned in said receptacles of said arms to pivotably mount said saddle to said receiver with said support member spaced proximally from said bottom surface of said channel.

12. The bone anchor assembly of claim 11, wherein said connectors define a pivot axis in said receptacles that is located proximally of said longitudinal axis of said connecting element and said support member is pivotal in said receiver to vary an orientation of said proximal support surface in said channel relative to said central longitudinal axis.

13. The bone anchor assembly of claim 12, wherein said support member includes a notch in at least one of said proximal support surface and a distal surface opposite said proximal support surface to facilitate flexing of said legs toward one another.

14. The bone anchor assembly of claim 12, wherein at least one of said connectors is movably engaged in a receptacle of said leg, said at least one connector being spring-biased outwardly from said leg.

15. The bone anchor assembly of claim 1, wherein:
said saddle includes a support member defining said proximal support surface, said support member extending between said opposite legs, said legs extending proximally from said support member and said opposite legs include a thread profile extending therealong;
said legs of said saddle are pivotally mounted to respective ones of said arms of said receiver; and
said engaging member is threadingly engaged to said thread profile of said legs of said saddle.

16. A bone anchor assembly, comprising:
a distal bone engaging portion and a receiver extending proximally from said bone engaging portion along a central longitudinal axis, wherein said receiver and said bone engaging portion form a fixed unitary structure and said receiver defines a channel extending therethrough;
a saddle mounted to said receiver in said channel, said saddle including a proximal support surface;
an elongated connecting element extending along a longitudinal axis, said connecting element extending through said channel and projecting from opposite sides of said receiver, wherein said longitudinal axis of said connecting element and said central longitudinal axis define a plane; and
an engaging member in contact with said connecting element to secure said connecting element against said proximal support surface of said saddle, wherein said saddle rotates only in said plane to align said proximal support surface with an orientation of said connecting element relative to said central longitudinal axis.

17. The bone anchor assembly of claim 16, wherein said receiver includes a pair of arms extending along said central longitudinal axis on opposite sides of said channel and a bottom surface extending along said channel between said pair of arms, wherein said channel opens at a proximal end of said pair of arms and said channel opens at said opposite sides of said receiver, said engaging member being engageable to said receiver through said proximal end opening of said channel.

18. The bone anchor assembly of claim 17, wherein:
said pair of arms include inner surfaces facing one another on opposite sides of said channel, said inner surfaces each include an arcuate slot portion formed therein that extends transversely to said central longitudinal axis between said opposite sides of said pair of arms, said inner surfaces each including a longitudinal slot portion therein extending from said proximal end of said respective arm to said arcuate slot portion of said respective arm; and
said saddle includes a support member defining said proximal support surface and a pair of legs extending from opposite sides of said support member, said legs being positioned in and slidably movable along a respective one of said longitudinal slot portions and said arcuate slot portions.

19. The bone anchor assembly of claim 17, wherein:
said receiver includes an elongate, arcuate slot formed in said bottom surface that extends in a direction between said opposite sides of said receiver, said arcuate slot including an enlarged distal portion and a narrower proximal portion extending between said distal portion and said channel; and
said saddle includes a support member defining said proximal support surface, said support member further including a distal surface opposite said proximal support surface and a fin extending distally from said distal surface that is positioned in and slidable along said arcuate slot of said receiver to vary an orientation of said proximal support surface in said channel in accordance with a position of said saddle along said arcuate slot.

20. The bone anchor assembly of claim 17, wherein:
said saddle includes a convexly curved distal surface opposite said proximal support surface, said distal surface including an elongate, arcuate slot formed therein; and
said receiver includes a post extending proximally from said bottom surface that is positioned in said arcuate slot of said saddle and said saddle is slidable along said post to vary an orientation of said proximal support surface in said channel in accordance with a position of said saddle along said post.

21. The bone anchor assembly of claim 17, wherein:
said saddle includes a generally U-shaped body with opposite legs and a support member extending between said legs, wherein said legs extend proximally from said support member and along opposite sides of said connecting element to a proximal end of said legs located proximally of said connecting;
a connector extending outwardly from said proximal end of each of said legs, said connectors extending in opposite directions from one another; and
said receiver includes a receptacle in each of said arms and said connectors are positioned in said receptacles of said arms to pivotably mount said saddle to said receiver with said support member spaced proximally from said bottom surface of said channel.
